(19) United States
(12) Patent Application Publication
(10) Pub. No.: US 2006/0068026 A1
(43) Pub. Date: Mar. 30, 2006

(54) Thermal electrochemical synthesis method for production of stable colloids of "naked" metal nanocrystals

(76) Inventors: Michael Z. Hu, Knoxville, TN (US); Clay E. Easterly, Knoxville, TN (US)

Correspondence Address: UT-Battelle, LLC;Office of Intellectual Property, One Bethal Valley Road, 4500N, MS-6258, Oak Ridge, TN, 37831, US

(21) Appl. No.: 11/154,973

(22) Filed: Jun. 16, 2005

Related U.S. Application Data
(63) Continuation-in-part of application No. 10/917,047, filed on Aug. 11, 2004.

(51) Int. Cl. A61K 33/38

(52) U.S. Cl. 424/618

(57) ABSTRACT

A method of making a colloidally stable suspension of naked metal nanocrystals includes the steps of: at least partly immersing a metallic sacrificial anode and a cathode into a body of essentially contaminant-free water, the metallic sacrificial anode that includes an essentially contaminant-free metal starting material for making nanocrystals; and applying a voltage potential across the anode and the cathode to form a colloidally stable suspension of naked metal nanocrystals composed essentially of metal from the metallic sacrificial anode.

### CROSS-REFERENCE TO RELATED APPLICATIONS

This patent application is a continuation-in-part of U.S. patent application Ser. No. 10/917,047 filed Aug. 11, 2004 by Clay E. Easterly, et al. entitled “Novel Treatment for Biological Warfare Pathogens”, the entire disclosure of which is incorporated herein by reference.

The United States Government has rights in this invention pursuant to contract no. DE-AC05-000R22725 between the United States Department of Energy and UT-Battelle, LLC.

### FIELD OF THE INVENTION

The present invention relates to high purity colloidal metal nanoparticles and methods of preparation thereof, and more particularly to stable suspensions of discrete, naked, metal nanocrystals and methods of preparation thereof.

### BACKGROUND OF THE INVENTION

Nanoparticles are elementary building blocks for controlled bottom-up assembly into nanostructured materials or next-generation nanodevices. The lack of sufficient stability (colloidal, chemical, etc.) of many nanoparticle preparations has impeded the development of real world applications of nanomaterials.

Metallic nanoparticles have in recent years received much attention as discoveries have been made into the various physical properties thereof. Potential applications include many and various devices and methods. Examples of such devices and methods include microelectronic devices (such as light emitting diodes and photovoltaic cells, for example), catalytic devices, antibacterial devices, magnetic devices, diagnostic biological probes, displayed devices, and optical fields such as surface-enhanced Raman spectroscopy, for example.

Subsets of nanoparticles include nanocrystals and nanoclusters. Nanocrystals are characterized as fully (at least 95%) crystalline by analytical methods such as transmission electron microscopy, while nanoclusters are not as highly crystalline, and may be fully amorphous.

Nanocrystalline Ag (Ag) has been demonstrated to have both anti-bacterial and anti-inflammatory properties. It has been successfully used in wound healing bandages that protect against bacterial infections, as an antimicrobial incorporated into athletic socks to reduce unwanted odors, and is being clinically tested in topical anti-inflammatory creams. Nanocrystalline Ag produced by various methods has been evaluated for its antimicrobial properties and differences in methods of production are found to result in various antimicrobial capabilities. However, such nanocrystalline Ag reported so far exist only as films and the nanoparticles are not in the dispersed nanosize (nominally ≦100 nm) form. Colloidal Ag is known as an effective eradicator of bacteria that may be antibiotic-resistant, and has been purveyed as a nutritional and or health supplement for many years. Organic stabilizers are used to maintain the stability colloids.

For some applications such as for ultrafast optical switching and polarizing filters, dispersed nanosize crystals are critical because quantum confinement properties only occur in each individual entity of a nanoparticle or nanocrystal, not in their aggregate forms.

Stability of preparations of metallic nanoparticles has been improved by utilization of surface coatings of colloidal “stabilizers” such as organic capping molecules (thiol or amine-based ligands/linkers for example), surfactants or polymer dispersants (polyvinyl alcohol (PVA) and polyvinyl pyrrolidone (PVP) for example), and citrate.

However, for many applications, organic stabilizer coatings contaminate the nanoparticle surfaces and thus degrade the desired properties and/or functionalities of the nanoparticle system. For example, uncoated, surface capping molecule-free (clean or “naked”) Ag nanocrystals (<10 nm) are needed for encapsulation in molecular self-assembly such as liposomes (˜100 nm diameter) to create a package that may be suitable for targeted therapy due to the antibacterial property of nanocrystal Ag. In addition, naked nanocrystals might show totally different properties from those capped nanocrystals since currently there is no knowledge about the possible adverse effect of organic surface molecules on properties. Capping molecules might very well block the surface active sites for catalysis. For sensing applications such as surface-enhanced Raman scattering that require analyte adsorption to the metallic nanoparticle surface, the presence of organic residue from the synthesis on the nanoparticle surface may cause a significant interference. Surface-bound residue or capping molecules from the synthesis increases the difficulty of doing chemistry at the surface. Therefore there is an unmet need for naked metal nanocrystals.

Prior methods of making colloidally stable suspensions of metal nanocrystals involve the use of organic molecules as reductants or stabilizers. A method is needed that can produce colloidally stable, discrete, naked nanocrystals less than 100 nm, especially less than 10 nm. Removal of stabilizing agent or organic residues from the nanocrystal surfaces is a difficult task and causes deleterious particle aggregation in tests of prior methods.

### OBJECTS OF THE INVENTION

Accordingly, objects of the present invention include methods of making colloidally stable, discrete, naked, metallic nanocrystals less than 100 nm in particle size, especially less than 10 nm in particle size. Further and other objects of the present invention will become apparent from the description contained herein.

### SUMMARY OF THE INVENTION

In accordance with one aspect of the present invention, the foregoing and other objects are achieved by a method of making a colloidally stable suspension of naked metal nanocrystals that includes the steps of: at least partly immersing a metallic sacrificial anode and a cathode into a body of essentially contaminant-free water, the metallic sacrificial anode that includes an essentially contaminant-free metal starting material for making nanocrystals; and applying a voltage potential across the anode and the cathode to form a colloidally stable suspension of naked metal nanocrystals composed essentially of metal from the metallic sacrificial anode.

In accordance with another aspect of the present invention, a composition of matter includes a colloidally stable suspension of naked metal nanocrystals having an average particle size of no more than 100 nm.

### BRIEF DESCRIPTION OF THE DRAWINGS

FIG. 1a is a schematic illustration of a thermal electrochemical synthesis reactor in accordance with the present invention having electrodes encapsulated with one U-shaped membrane tube with a center barrier.

FIG. 1b is a schematic illustration of a thermal electrochemical synthesis reactor in accordance with the present invention having electrodes encapsulated with two separate membrane tubes with the bottoms sealed or tied.

FIG. 1c is a schematic illustration of a thermal electrochemical synthesis reactor in accordance with the present invention having electrodes encapsulated in a single, large membrane tube.

FIG. 2 is a schematic illustration of contemplated Ag nanocrystal formation mechanisms in a thermal electrochemical synthesis reactor.

FIG. 3a is a transmission electronic (TEM) image of typical Ag nanocrystals (≦10 nm) in accordance with the present invention.

FIG. 3b is an enlarged transmission electronic (TEM) image of the Ag nanocrystal shown at arrow A in FIG. 3a.

FIG. 3c is an enlarged transmission electronic (TEM) image of the Ag nanocrystal shown at arrow B in FIG. 3a.

FIG. 4 is a graph showing temperature and reaction time effects on nanocrystal formation in accordance with the present invention.

FIG. 5a is a graph showing voltage effect on UV/vis absorption peak position (i.e., nanocrystal size) in accordance with the present invention.

FIG. 5b is a graph showing voltage effect on absorbance (i.e., nanocrystal concentration) in accordance with the present invention.

FIG. 6 is a graph showing UV/vis absorbance changes during nanocrystal formation process in accordance with the present invention.

FIG. 7 is a graph showing correlation of dynamic light scattering (DLS) particle size with reaction time and temperature in accordance with the present invention.

FIG. 8 is a graph showing correlation of nanocrystal size by DLS and UV/vis absorption peak position in accordance with the present invention.

FIG. 9a is a graph showing data for particle size vs. reaction time in accordance with the present invention.

FIG. 9b is a graph showing data for particle concentration (absorbance) vs. time in accordance with the present invention.

FIG. 9c is a graph showing data for absorption peak position vs. particle size in accordance with the present invention.

FIG. 9d is a graph showing data for comparison between a larger (150 ml) reaction vessel and a smaller (50 ml) reaction vessel in accordance with the present invention.

For a better understanding of the present invention, together with other and further objects, advantages and capabilities thereof, reference is made to the following disclosure and appended claims in connection with the above-described drawings.

### DETAILED DESCRIPTION OF THE INVENTION

The present invention relates to a membrane-assisted thermal electro-chemical (TEC) method of producing colloidally stable aqueous suspensions (or sols) of discrete, naked, metal nanocrystals having a particle size in a range of about 1 nm to no more than 100 nm, preferably about 1 nm to no more than 50 nm, more preferably about 1 nm to no more than 20 nm, most preferably about 1 nm to no more than 10 nm. The metal nanocrystal colloidal sols are preferably “naked”, having essentially no organic molecules (surface capping ligands, surfactants and/or polymer dispersants, for example) adhering to the surfaces thereof or in the bulk aqueous solution background, other than trace amounts as impurities.

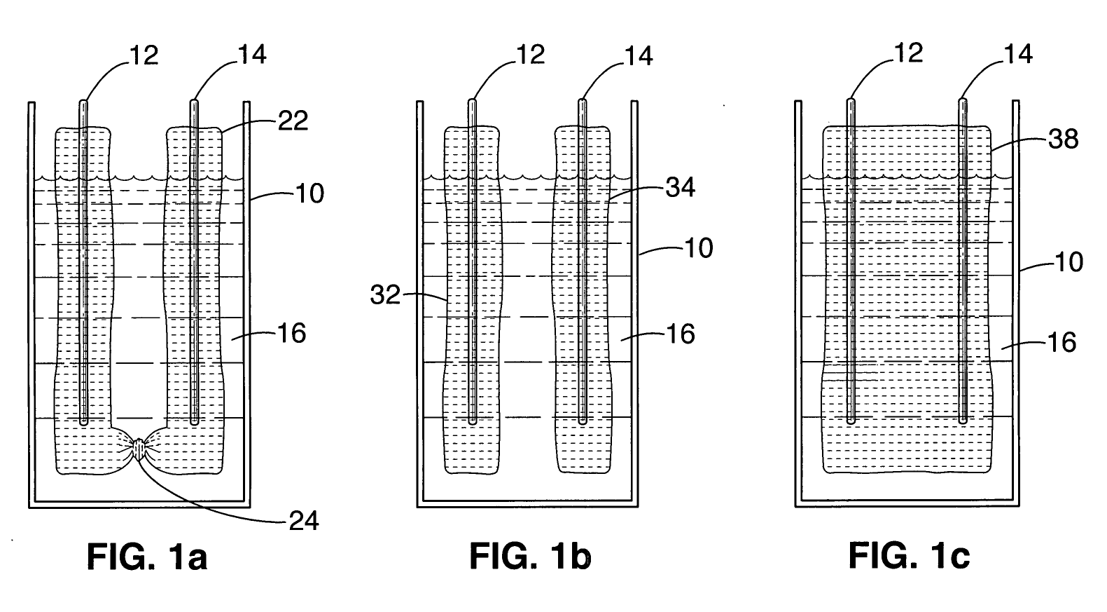

Referring to FIGS. 1a, 1b, and Ic, the method of the present invention involves an electrolytic cell 10 having an anode 12, cathode, 14, and an aqueous process medium 16 which is usually deionized water. The anode 12 and cathode 14 are spaced apart; the spacing can be as little as 1 mm or as much as 100 mm; electrode spacing is not especially critical to the invention per se, although it plays a part in optimizing the process.

The anode 12 comprises the metal source that is to be converted into naked nanocrystals; the cathode 14 can be the same or a different material. Although Ag is predominantly used in the following description of the present invention, many other metals can be used in the practice thereof. Suitable Anode metals include, but are not limited to Ag, Au, Pd, Pt, Cu, Ru, Os, Ti, Fe, Cr, Co, Rh, Ni, Cd, Zn, Al, alloys of any of the foregoing, and any other metal electrochemically reducible from an aqueous solution to a stable, crystalline, metallic form.

An AC or DC voltage, preferably DC, usually in the range of 1-50 V, but not critically so, is applied to the electrodes. A preferable range of voltage is 10-30 V, in terms of the rate of nanocrystal formation and control of nanocrystal size and size distribution. Optimal voltages are generally dependent on the anode materials and the geometry of the apparatus. Parametric data can be developed relating to various parameters such as, for example, size, absorbance peak, etc. as a function of voltage and other experimental variables. Such data can be used to optimize the voltage and other method parameters.

When electric current passes through the Ag anode, some Ag atoms at the interface with water will lose an electron and become ions. In this electrochemical process, some of the Ag ions in close proximity to the anode accept electron from the current passing through and reduce to metallic atoms, which attract each other by Van Der Waal's forces of attraction and thus form small metallic nanoclusters. Overall, the electric current flow causes Ag⁰ (metal) and Ag⁺ (ions) to migrate from the electrode into the deionized water.

The method can be enhanced by using tubular dialysis membranes (DM) around the electrodes 12, 14. FIG. 1a shows a tubular membrane 22 over both electrodes 12, 14, optionally having a cinch 24 in the middle thereof. FIG. 1b shows separate membranes 32, 34 over respective electrodes, 12, 14. FIG. 1c shows a single membrane 38 enclosing both electrodes 12, 14. The reaction vessel, membrane(s), and other parts of the apparatus should be free of contaminants in order to promote the purity and desired size distribution of the product.

The purpose of membrane(s) as described herein is to enclose over-grown crystals within the membrane tubular sac(s), only allowing Ag ions or small clusters to pass through the membrane pores. The membrane effectively narrows the range of size distribution of nanocrystals, avoiding formation of large size aggregate particles in the bulk medium outside the membrane due to fall off of over-grown agglomerative deposits on/near either or both electrodes. The membrane should separate the electrodes from the bulk liquid and preferably from each other.

Dialysis membranes are suitable for carrying out the present invention. Other suitable membranes include ceramics, polymers, and other organic and/or inorganic membranes having suitable pore-size and structure as is evident from the present description.

The geometry of the electrodes can be in various forms and geometry such as wire, flat sheets, etc. Corresponding to different electrodes, different shapes of membranes can be used to separate the electrode from the bulk solution.

Pore-size of a membrane is generally designated by a nominal molecular weight cut-off (MWCO) value. The method of the present invention is operable using a membrane having a MWCO in the range of 1,000 to 50,000, preferably in the range of 2,000 to 10,000, more preferably in the range of 2,500 to 7,000, still more preferably in the range of 3,000 to 5,000, most preferably about 3,500.

Without the use of membranes to encapsulate the two electrodes, black colored agglomerate deposits will typically accumulate with time around the anode, while white colored deposits will grow around the cathode. The agglomerate deposits grow in a fractal (tree-like) fashion between the two electrodes and they break and fall off eventually into the solutions. Meanwhile, the color of the initial water turns into bright golden yellow, an indication of small (≦10 nm) Ag nanocrystals. A typical sample contains not only small nanocrystals, but also some large nanocrystals (tens of nanometers to 100 nm) plus some small numbers of micometer-sized aggregate particles. Filtration with 0.2 μm microdisc syringe filters usually remove the large particles and reduce the size distribution, producing nanocrystals in tens of nanometers size range.

The present invention produces aqueous sols containing metallic colloid Ag nanocrystals. In contrast to conventional electrochemical methods, which, although AC or DC voltage and Ag wire electrodes are employed, are in fact electrolysis processes that produce mostly ionic Ag.

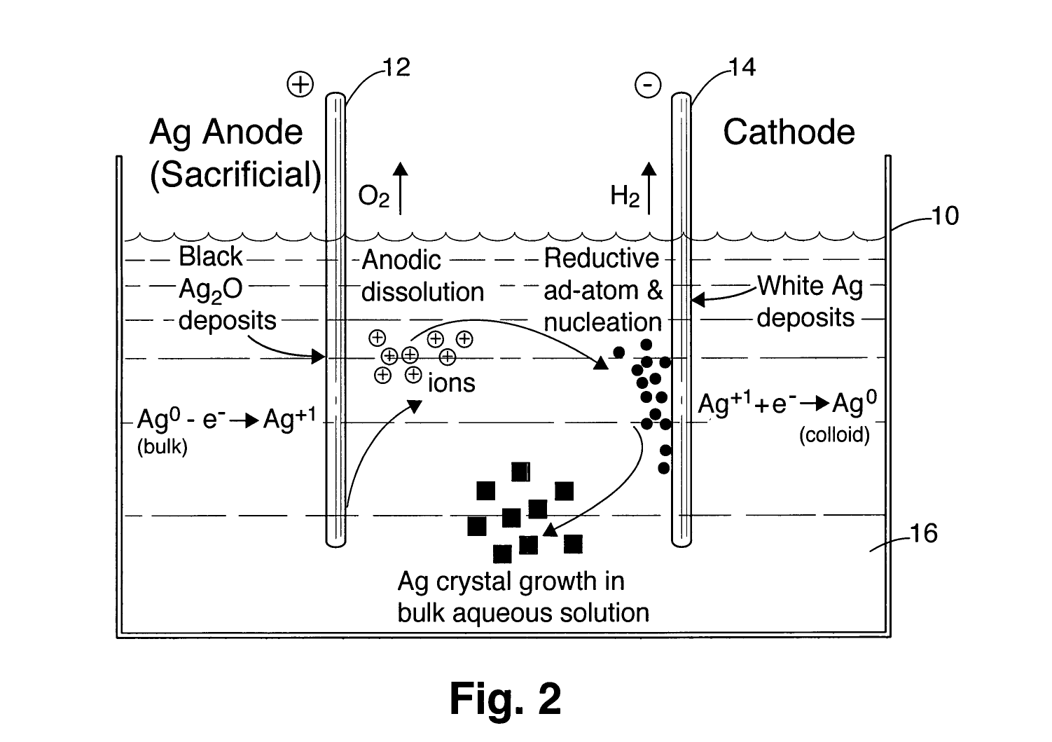

The electrochemical process may involve multiple mechanisms, which are schematically illustrated in FIG. 2. It is generally assumed that water ionizes to H⁺ and OH⁻, and that the H⁺ (in the form of the hydronium ion, H₃O⁺) migrates to the cathode (i.e., “−” Ag electrode), where it is reduced to hydrogen gas, which is liberated. The electrons taken from the cathode are replaced at the anode (i.e., “+” sacrificial Ag electrode), where Ag metal is oxidized and goes into solution as Ag⁺. Ag cations from the anode then migrate to the cathode where reduction occurs with the formation of metallic Ag atoms. The metal Ag atoms them form clusters (nanocrystals) in the vicinity of cathode. The clusters migrate away from the cathode and may grow further to form insoluble nanocrystals in the bulk solution, depending on the cluster-ion or cluster-cluster interactions. In addition, the hydrogen evolved from the cathode surface due to electrolysis of water could also contribute to the reduction of Ag ions into metallic Ag. The discussion of possible mechanisms is to assist generally in the optimization of the present invention for achieving desired results for various applications.

The process temperature is an important parameter that may affect the anode dissolution rate, the reactive nanocluster formation rate, the Ag crystallization rate and nanocrystal size, and even possibly the nanocrystal aggregation. The method of the present invention is operable at any temperature in the range of 10° C. to 100° C., preferably 90° C. to 99° C. at atmospheric pressure. The method of the present invention is also operable at any temperature greater than 100° C. at a pressure sufficient to maintain the water in the liquid state.

Various specific examples of the present invention are described hereinbelow in a discussion of a series of experiments wherein various process parameters were tested.

### EXAMPLES

Clean 150 ml beakers and other types of glass vials (e.g., small reaction vessels and scintillation vials) were used as reaction vessels, with magnetic stir bars to provide agitation. Reactor tops were manufactured using appropriate sizes of black rubber stoppers with holes drilled therein at varying spacing to control the inter-electrode spacing. Electrodes comprised clean, 14-gauge round 99.99% pure Ag wire cut to 9 cm length and rolled straight.

Spectra/Por 3 Dialysis membrane (DM) with 3,500 molecular weight cut-off (MWCO) was placed around the two Ag electrodes to reduce contamination of samples and deter larger Ag crystals (white to brown color, deposited around the ground electrode) or Ag oxide (black color, deposited around the positive electrode) from entering the bright yellow nanocrystal sol. DM membranes were cleaned (to remove metals and sulfur impurity from the membranes) with the following procedure before they were installed over the electrodes. Membranes (with 15 centimeter diameter) were prepared in bulk over a four hour time period using various heated washes of sodium carbonate, EDTA, sodium sulfite, sulfuric acid, deionized water and 0.02 cm filtered deionized water. Bulk membranes were then stored in 0.02 cm filtered deionized water in a refrigerator, to keep samples from contamination by bacteria.

More specifically, membrane preparation and final rinses were performed using filtered DI water. Metals were removed by light boiling in 1% sodium carbonate, 10 mM EDTA DI water 1 hour followed by 2 stirred rinses in DI water (30 min each at 80-100° C.). Sulfur was removed by cleaning in stirred bath with 0.03% Sodium Sulfite for one minute at 80° C. followed by washing with stirred bath 2 minutes in deionized water at 60° C. followed by 1 minute washing with Sulfuric acid to 1.6×10⁻⁴ concentration at 60° C. The DM was then rinsed 3 times with DI in stirred bath for 20 minutes each (80-100° C.). Two rinses with filtered deionized water completed the preparation. Such meticulous preparation is not critical to the invention, but provides the highest purity product.

Deionized water was filtered and placed in the reaction containers. Clean surgical gloves were worn to fit the membrane onto pipette tips and fill the membrane tube with 0.02 μm filtered deionized water using pipettes. Electrodes were carefully placed in the filled membranes and secured using silicon tubing. The membrane-covered electrodes were assembled into the stoppers which were inserted into the deionized water in the reaction container.

The reaction container was partially immersed in a heated silicone oil bath to maintain a selected temperature in the reactor vessel. A voltage-controllable D.C. power supply provided selected voltage to the electrodes to cause electrical current to flow through the reaction solution. Before voltage was applied, the water temperature for each sample was allowed to equilibrate with the oil bath (20 min for the small reaction vessels and 40 min for the 150 ml beaker). Process parameters such as voltage, temperature, and time were then varied for different samples. Samples were immediately collected after each reaction run and stored in either small reaction vessels or scintillation vials. Once cooled to room temperature, samples were analyzed using a dynamic light scattering (DLS) device and a UV/vis spectrophotometer.

Process parameters such as temperature, DC voltage, reaction time, spacing electrodes, reaction solution composition, and enclosure of DM membrane tubes around electrodes were all found to have significance on the Ag nanocrystal formation. Improper conditions (temperature, voltage, in particular) could result in undesirable product such as, for example, precipitated solution containing settled solid aggregates, a copper-colored solution likely containing overly large size particles, or a colorless solution likely indicating insignificant particle formation.

Dialysis membrane was not used in the original sixteen runs conducted; twenty-one runs were conducted with dialysis membrane to determine conditions needed for the production of nanocrystals. Results of many early samples are not included due to copper color or clear color of solution indicating the presence of undesired products. By properly controlling conditions as described herein, (temperature, voltage, electrode spacing, and adding a constantly stirring stir-bar to the reaction), a golden bright yellow color of solution was achieved, indicating existence of small Ag nanocrystals of ≦10 nm.

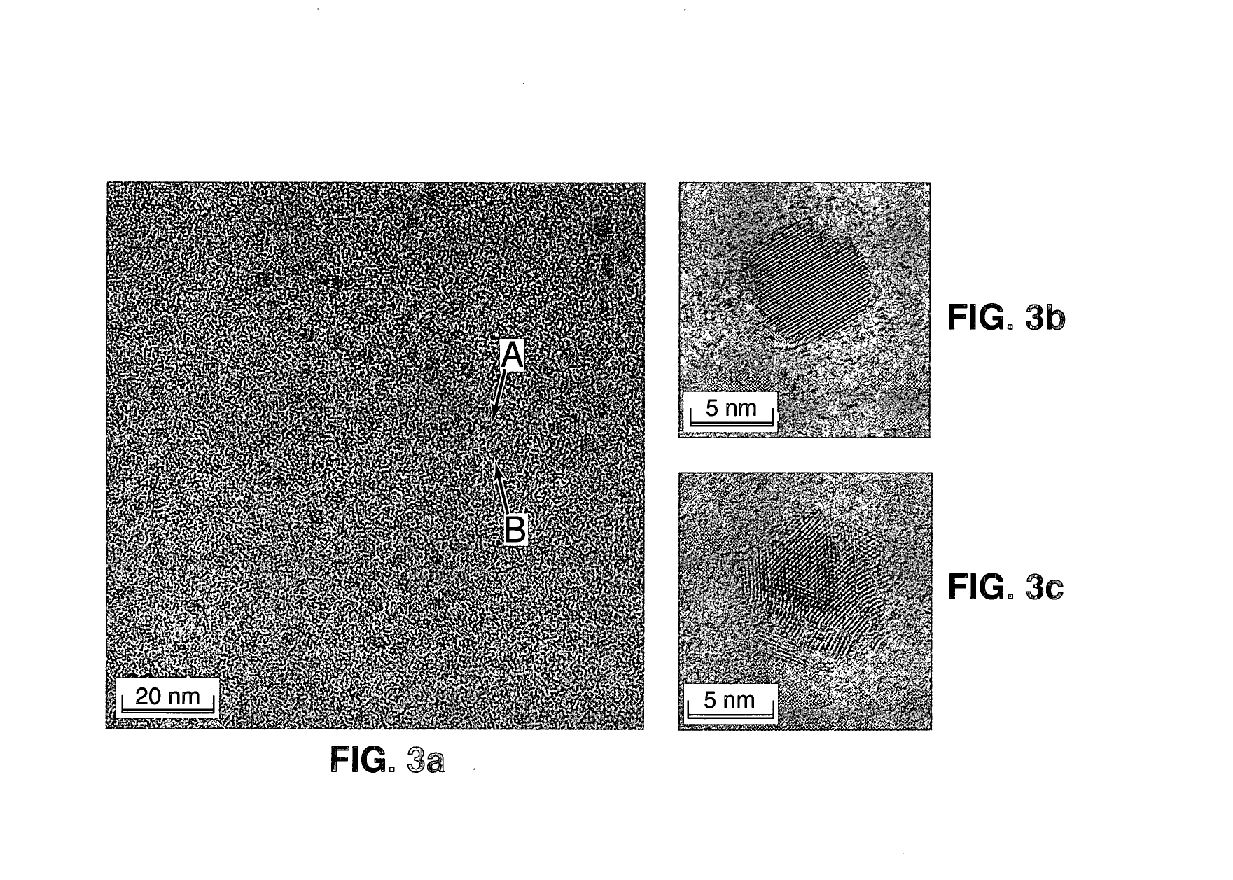

During a typical run of synthesis, first visible signs of yellow color would appear in the dialysis membrane tube, and as the membrane would begin to go from golden yellow to brown to black (visible bubbles would occasionally be seen on the electrode) the solution would visibly turn yellow. By the end of the run, dialysis membrane would be black and the bulk solution would be yellow. If the electrodes were in contact with the dialysis membrane a Ag color would appear in the dialysis membrane. A typical transmission electron microscope (TEM) image for nanocrystals from the yellow solutions is shown in FIG. 3a, with areas indicated by arrows A and B shown enlarged in FIGS. 3b and 3c.

Table 1 displays the runs that have produced bright yellow color of solutions, their various reaction times, temperatures, electrode spacing, measured DLS effective diameters with poly distributions (a measure of size uniformity, with smaller numbers indicating more uniformity), kcns/s (thousands of counts per second) UV/vis absorbance wavelength peaks, wavelength of maximum UV/vis absorbencies, and absorption peak height, with higher numbers indicating greater concentration of Ag.

TABLE 1

| | Duration of | | DLS Effective | | Scattering | Electrode | Abs. Peak | Peak |
|---|---|---|---|---|---|---|---|---|
| Sample | DC Voltage | Temp | Diameter | Poly | Intensity | Spacing | Wavelength | Height |
| No.* | (min) | (° C.) | (nm) | Dispersity | Kcns/s | (cm) | (nm) | abs. |
| DM-22 | 40.00 | 76 | 1.3 | 0.2783 | 8.230 | 1.1 | 411-417 | 0.181 |
| DM-23 | 80.00 | 76 | 69.7 | 0.5856 | 22.833 | 1.1 | 419 | 0.168 |
| DM-24 | 59.50 | 76 | 11.2 | 0.4212 | 14.163 | 1.1 | — | — |
| DM-25 | 40.00 | 76 | 4.0 | 0.2059 | 9.576 | 1.1 | 415-417 | 0.167 |
| DM-26 | 40.00 | 76 | 1.5 | 0.2593 | 8.514 | 1.1 | 416-420 | 0.187 |
| DM-27 | 40.00 | 86 | 1.6 | 0.2665 | 13.464 | 1.1 | 416-420 | 0.251 |
| DM-28 | 60.00 | 86 | 59.1 | 0.5012 | 27.856 | 1.1 | 415-422 | 0.144 |
| DM-29 | 80.50 | 86 | 67.3 | 0.5319 | 30.754 | 1.1 | 431-436 | 0.307 |
| DM-30 | 40.00 | 97 | 33.8 | 0.4545 | 33.765 | 1.1 | 428-431 | 0.385 |
| DM-31 | 60.00 | 97 | 66.6 | 0.4552 | 31.016 | 1.1 | 425-426 | 0.691 |
| DM-32 | 60.00 | 86 | 25.5 | 0.5176 | 23.261 | 1.1 | 422 | 0.424 |
| DM-33 | 70.00 | 97 | 63.8 | 0.2557 | 132.025 | 1.1 | 431 | 1.625 |
| DM-34 | 40.00 | 97 | 59.6 | 0.3904 | 42.308 | 1.1 | 420-426 | 0.278 |
| DM-35 | 40.00 | 97 | 37.6 | 0.5321 | 18.565 | 1.1 | — | — |
| DM-36 | 40.00 | 97 | 10.8 | 0.4292 | 10.332 | 1.1 | 419-423 | 0.258 |
| DM-37 | 40.00 | 97 | 29.2 | 0.5214 | 17.425 | 1.1/1.2 | 417&419 | 0.252 |
| DM-39 | 80.00 | ˜60 | 16.2 | 0.5092 | 7.561 | 1.1 | 410-417 | 0.088 |
| DM-42 | 80.00 | 76 | 71.5 | 0.5129 | 11.233 | 1.1 | 413-419 | 0.102 |
| DM-43 | 60.00 | 76 | 10.0 | 0.3975 | 8.079 | 1.1 | 415-416 | 0.207 |
| DM-44 | 80.00 | 86 | 13.1 | 0.4418 | 11.462 | 1.1/1.2 | 418-422 | 0.141 |
| DM-45 | 39.30 | 86 | 16.6 | 0.4460 | 8.803 | 1.1 | 414-417 | 0.334 |
| DM-46 | 60.00 | 86 | 29.9 | 0.4954 | 10.795 | 1.1 | 416-419 | 0.296 |
| DM-47 | 22.00 | 86 | 1.4 | 0.2744 | 6.237 | 1.1 | 411-416 | 0.217 |
| DM-48 | 23.00 | 86 | 110.4 | 0.3341 | 33.061 | 1.1 | 414-416 | 0.234 |
| DM-49 | 40.00 | 84 | 45.4 | 0.5390 | 10.956 | 2.0 | 409-413 | 0.127 |
| DM-50 | 40.00 | 84 | 46.8 | 0.4349 | 29.676 | 2.0 | — | — |
| DM-51 | 58.00 | 84 | 31.7 | 0.4971 | 17.277 | 2.0 | 420-422 | 0.555 |
| DM-52 | 40.00 | 84 | 0.7 | 0.2054 | 4.814 | 2.0 | 411-419 | 0.134 |
| DM-53 | 80.00 | 84 | 72.1 | 0.3457 | 27.810 | 2.0 | 413-414 | 0.753 |
| DM-54 | 41.17 | ˜100 | 69.2 | 0.4117 | 33.737 | 2.1 | 416-418 | 0.869 |
| DM-55 | 62.50 | ˜101 | 37.1 | 0.3925 | 41.828 | 2.1 | 434-437 | 1.004 |

*Sample Nos. DM-38, DM-40, and DM-41 are omitted because the membrane slipped off during the runs and so no information was gathered.

A systematic study to look at three temperatures and three times (at a fixed applied DC voltage, electrode spacing, and reactor arrangement) was then conducted to gain initial insight into the effects of these variables on the synthesis. Two size reactor vessels/containers are used for study. Repeated runs were conducted to look at reproducibility an can be viewed in Table 1 runs DM-22 through DM-49. DM represents each run was conducted with dialysis membrane tube (tied a knot at center) enclosed around electrodes. DM-22 through DM-48 were synthesized in small reaction vessels containing 3.8 mm diameter DM membrane tube and 1.1 cm electrode spacing with 10.0 volts DC.

In an attempt to improve reproducibility, results DM-49 through DM-55 were synthesized in 150 ml beakers, containing 11.5 mm diameter DM membrane tubes and 2.0 cm electrode spacing with 30.0 volts DC. All DLS information is an average of at least three measurements taken repeatedly from the same sample. Temperature was measured during a separate run with no current flowing through the sol.

Table 2 shows DLS measurements of samples DM-24 and DM-26. DM-26 was measured at various times over the course of a month to determine stability of solution. Clearly within a month the nanocrystal sample (<5 nm) is quite stable with little size increase.

TABLE 2

| | | | | Effective | | |
|---|---|---|---|---|---|---|
| | | Process | Temp | Diameter | Poly- | |
| Sample | Day | Time (min) | (o C.) | (nm) | Dispersity | kcns/s |
| DM-24 | 1 | 59.50 | 76 | 11.2 | 0.4212 | 14.163 |
| | 13 | | | 13.3 | 0.4594 | 10.268 |
| DM-26 | 1 | 40.00 | 76 | 1.5 | 0.2593 | 8.514 |
| | 8 | | | 3.4 | 0.3463 | 8.907 |
| | 13 | | | 4.8 | 0.4295 | 8.417 |
| | 29 | | | 5.2 | 0.3936 | 10.697 |

In several separate trials of concentrating the as-prepared nanocrystal solutions, heating-assisted evaporation in appropriate glass containers was effective to deepen the solution yellow color without increasing nanoparticle size significantly (per DLS measurements).

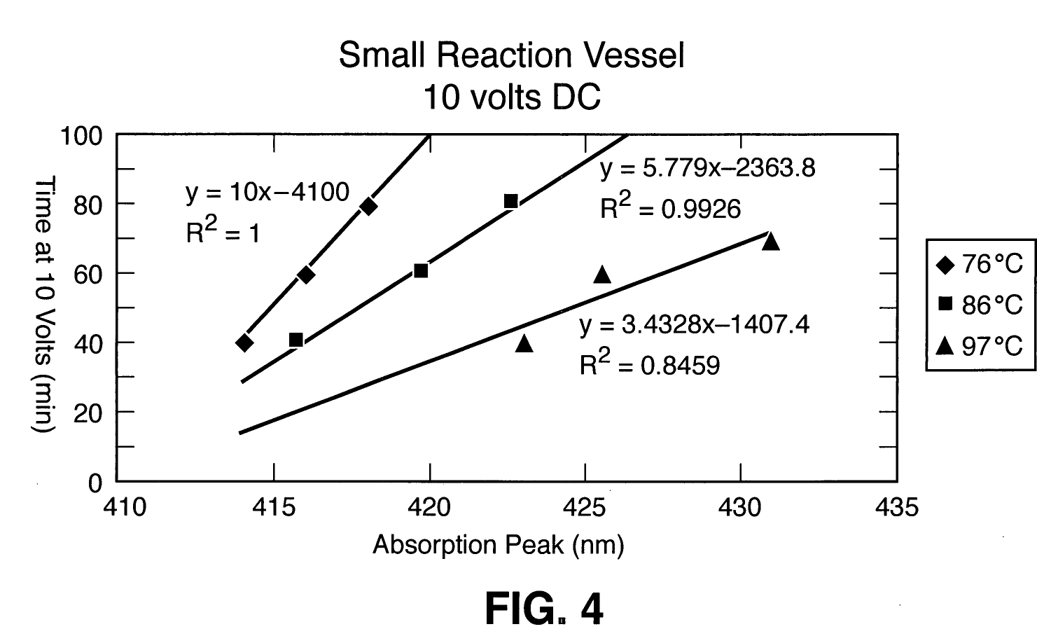

Data analyses in small vessels are shown in FIGS. 4-7. FIG. 4 shows the correlation between absorption peak positions of the UV/vis spectrum and reaction times under three temperature conditions (76, 86, and 97° C.). At each temperature, the correlation is linear. With increasing reaction time the wavelength (nm) of the absorption peak position increases (i.e., red shift), indicating particle size increases with time. Note that blue shift is the peak shift toward smaller wavelength due to the decrease of nanosize crystals. This is well known phenomena due to so-called size related quantum effect. With increasing temperature, the red shift is larger. Within the same reaction time, the red shift at lower temperature (76° C.) is less that the one at higher temperature (97° C.), indicating that higher temperature causes a faster crystal size growth.

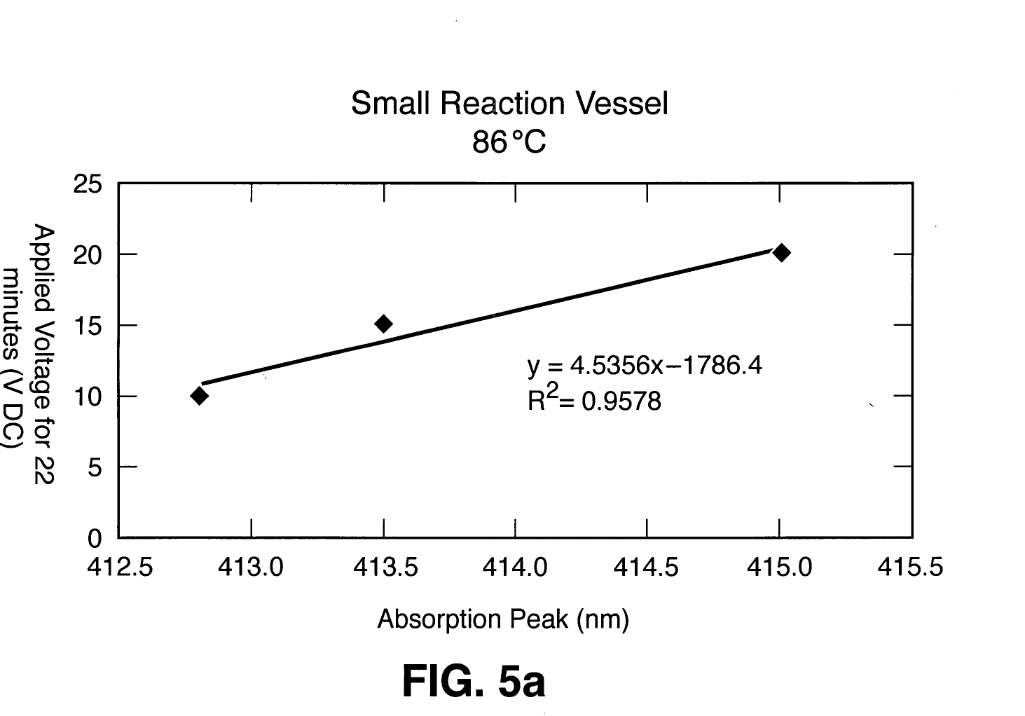

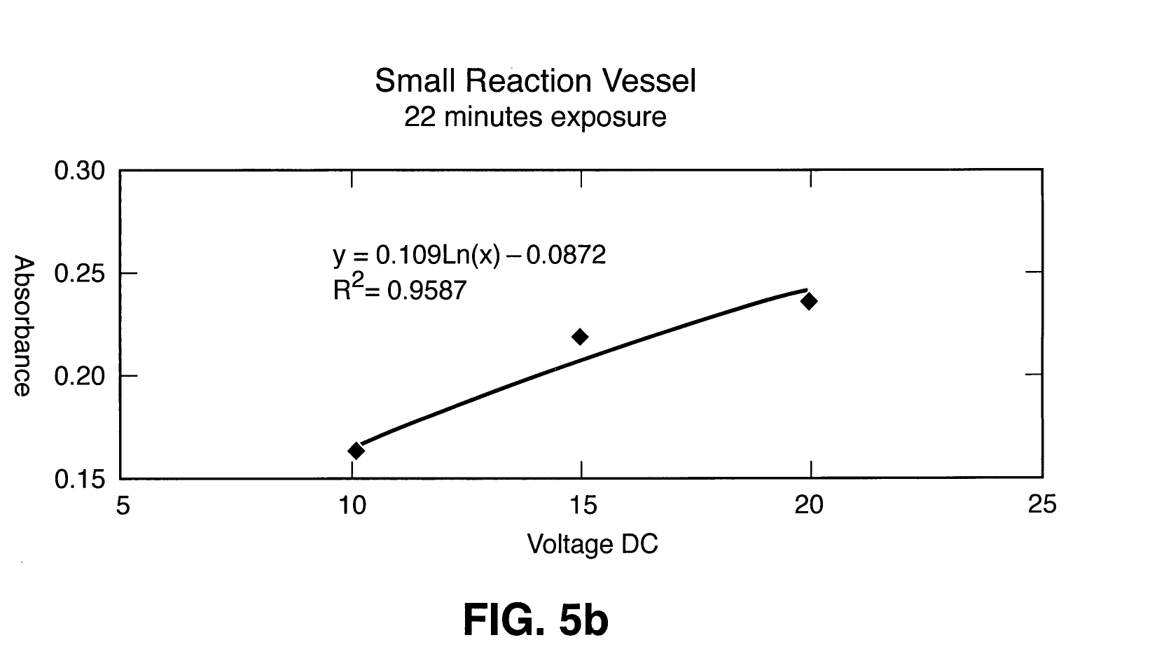

Voltage effects on the absorption peak position are shown in FIGS. 5a and 5b. Under the same conditions, higher voltage tends to produce nanocrystal samples giving higher absorption peak position, indicating that higher voltage causes a faster growth of nanocrystals. Higher voltage also leads to higher absorbance in the sample, indicating higher number concentration of nanocrystals.

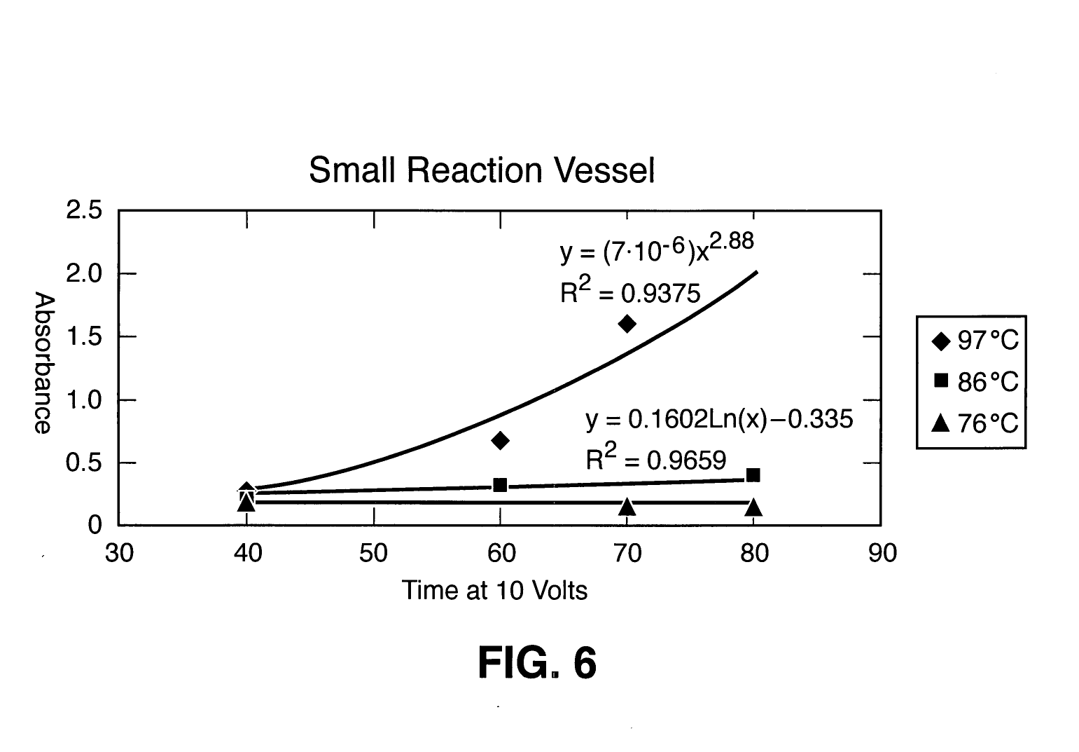

FIG. 6 shows the absorbance changes with the reaction time under three temperature conditions. There is a general trend of absorbance increase with reaction time, indicating the nanocrystal concentration increase with time. Such increase is particularly significant at higher temperature (such as 97° C.).

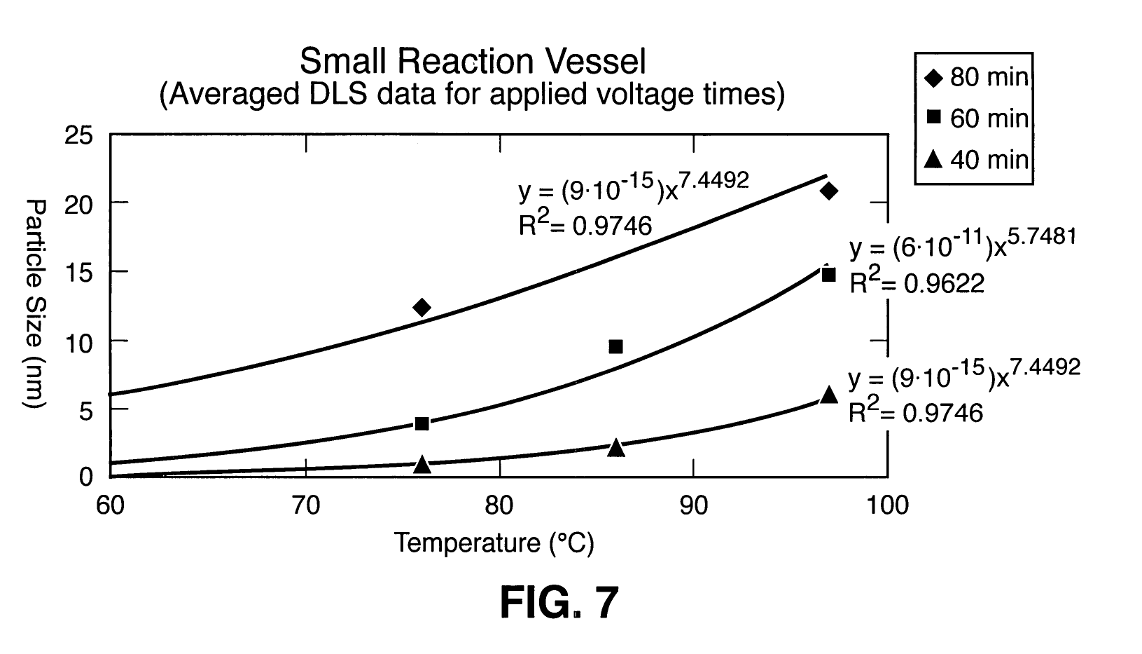

Particle size measured by the DLS is shown in FIG. 7. Clearly, higher temperature and longer reaction time will produce larger size nanocrystals. This trend of effect is consistent with the UV/vis measurements as shown in FIG. 4.

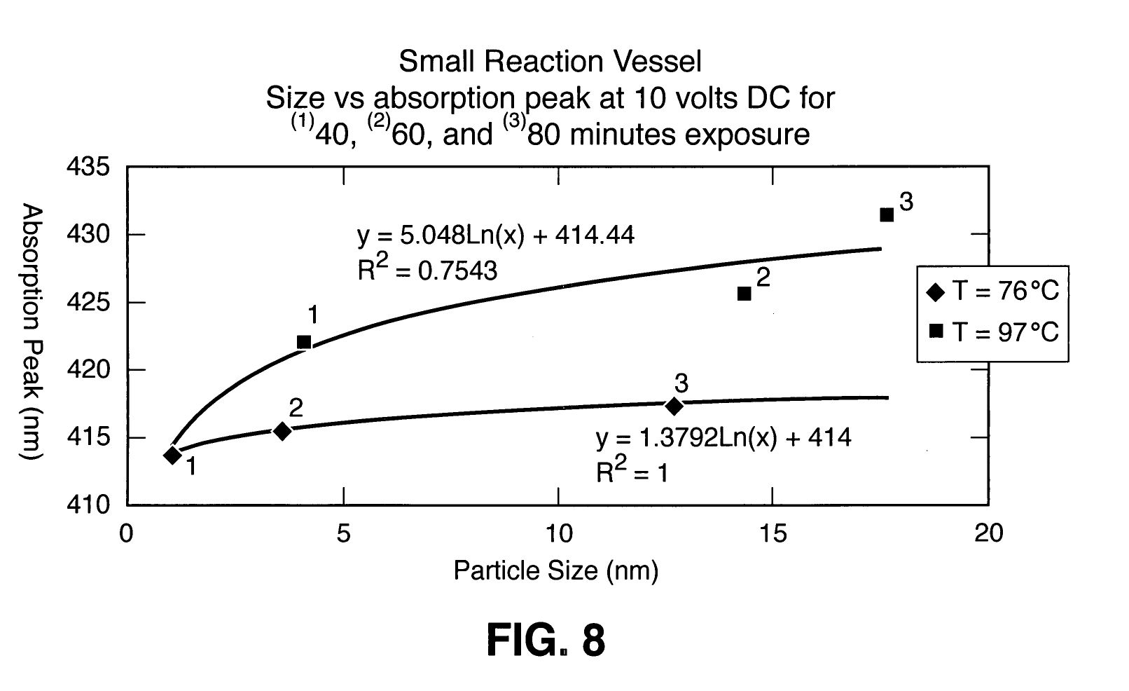

FIG. 8 shows the correlation between nanoparticle size (measured by DLS) and the absorption peak positions (measured by UV/vis spectrometer) of the nanocrystal sol samples at two different reaction temperatures (76° C. and 97° C.). Decrease of particle sizes (from ˜20 nm to 1 nm) correspond to a “blue shift” (i.e., absorption peak position decrease from ˜430 nm to 414 nm). This is in general consistent with the literature observations. In addition, it appears that nanocrystals obtained at higher temperature tend to give a larger wavelength absorption peak position that those obtained at lower temperature, in indicating possible effect of parameters other than size (e.g., particle shape or morphology) on the quantum mechanical properties of the nanocrystals.

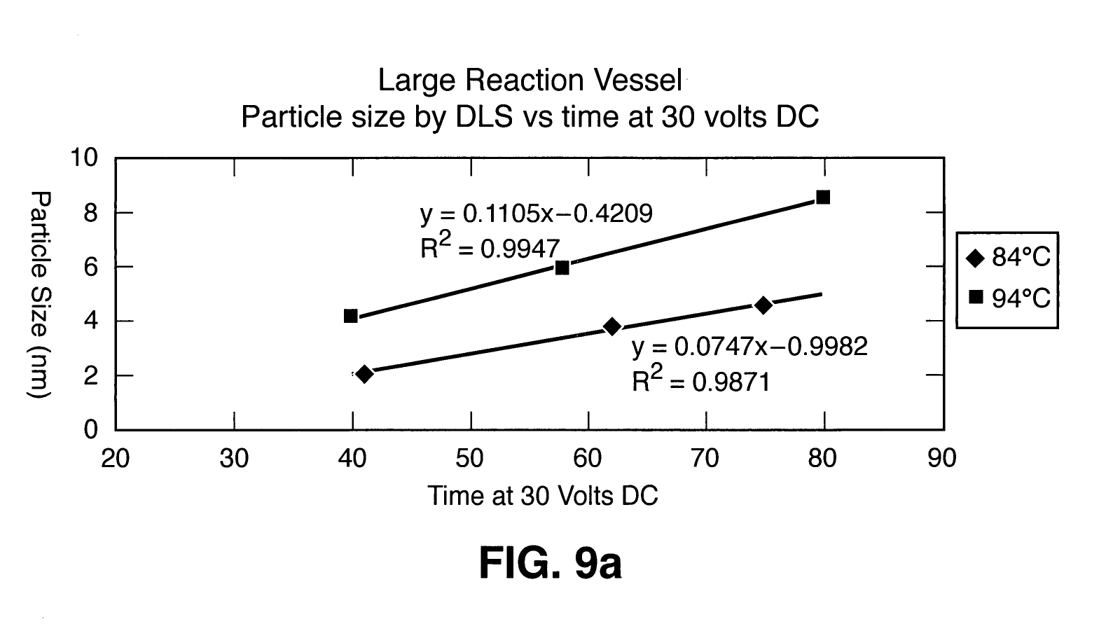

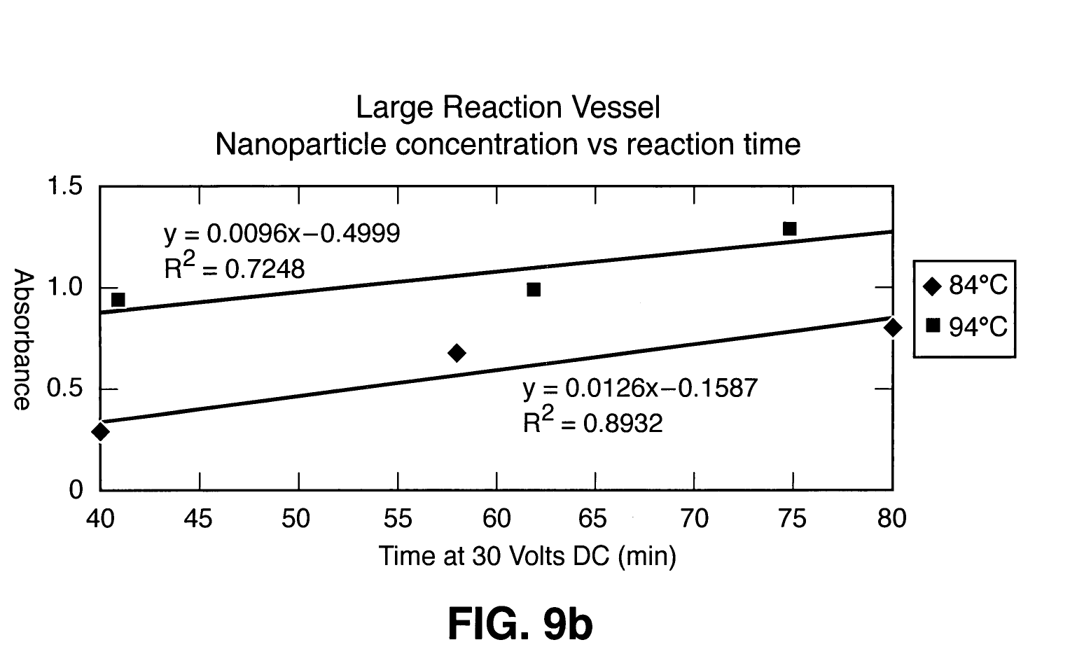

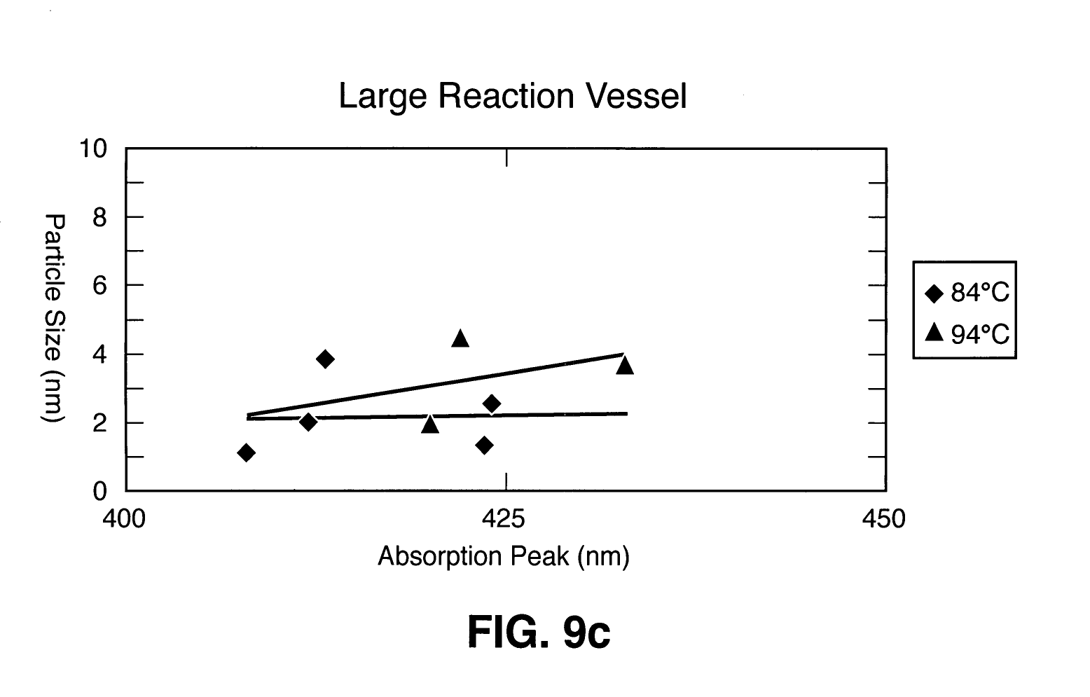

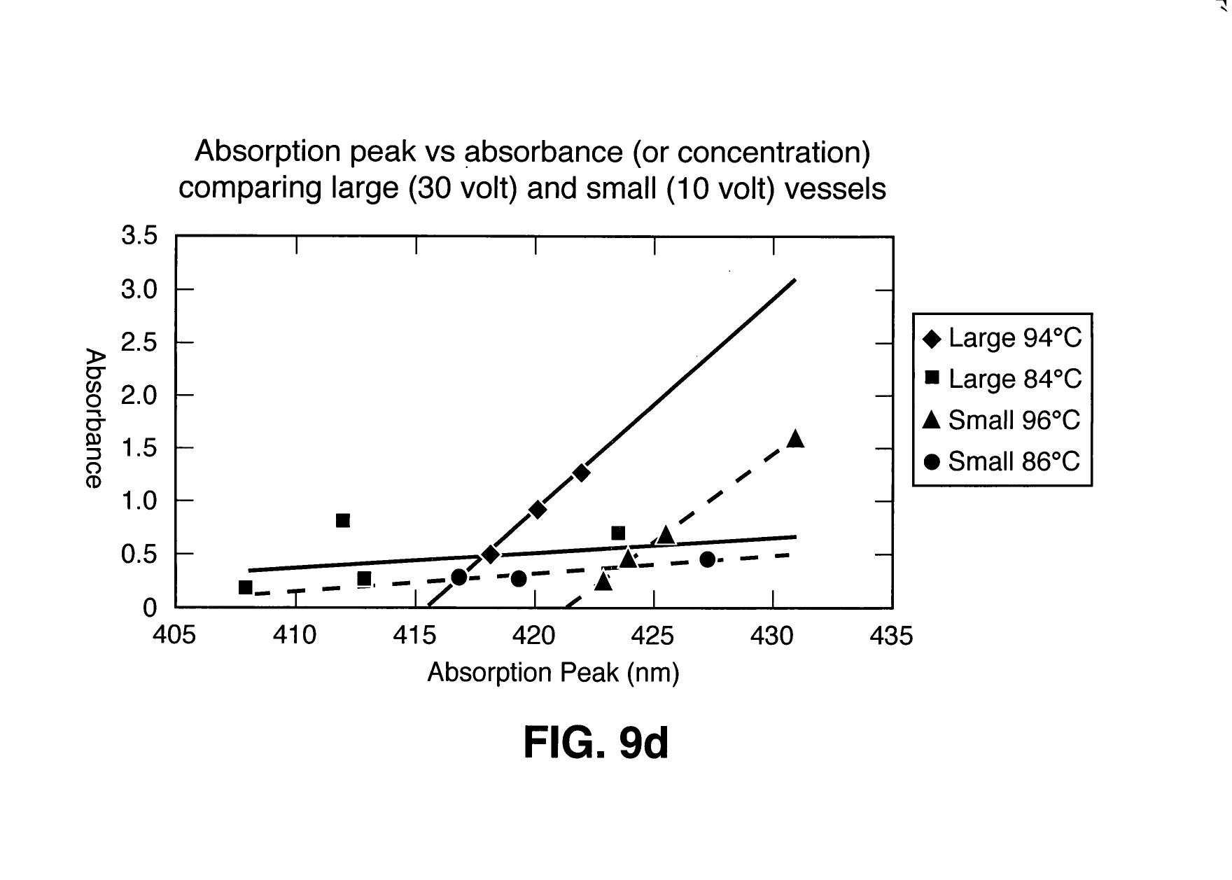

Data analyses are shown in FIGS. 9a-9d. FIG. 9a shows the general trend of nanocrystal size increase with the reaction time. FIG. 9b shows the UV/vis absorbance (an indicator of particle number concentration) increase with reaction time. FIG. 9c displays the correlation between the particle size and the absorption peak position. Finally, FIG. 9d compares the data between the large vessel and the small vessel.

In addition, other additive agents (such as PVA, poly vinyl alcohol) in the reactive solutions may affect significantly on the nanocrystal formation kinetics and mechanisms. We have observed that presence of PVA (concentration range: 1 to 100 ppm) accelerates the nanocrystal nucleation and growth and produces highly concentrated suspensions of nanocrystals. In a small reaction vessel with 5 ppm PVA at 98° C. and electrode spacing of 1 cm, no evidence of reaction at 1 volt for ½ hr. At 10 volts, evidence in 5 ppm PVA was seen in 8 minutes.

It is known from the literature that the critical radius r_{crit} of clusters of adatoms (prior to aggregated nanoparticle powder formation) at the cathode interface is given by the equation [Reetz and Helbig, 1994]:
r_{crit}=2M(/(nFΘΔ)

where M=molecular weight, (=surface tension, n=valence, F=Faraday constant, Θ=over-potential (which is defined as the deviation of the reduction potential from the equilibrium potential), and Δ=density of the cluster. Accordingly, r_{crit} is inversely dependent on the over-potential Θ, which in turn is directly related to the current density. This equation predicts a cluster size range r_{cit}=0.5-73 nm (e.g., choosing M=107.9; n=1; Δ=12 g/cm²; (=2±1 N/m, and Θ=50-10000 mV). Per our TEM examination of our Ag nanoparticle samples, we only see discrete (essentially single, not agglomerated) crystalline particles with size in the predicted range. This indicates that the synthesis condition effectively prevents metal nanocrystal agglomeration into precipitative powder solids. In fact, discrete Ag nanocrystals are very well dispersed in the solution to form a stable yellow/gold colored sol. In an unexpected contrast to the literature method, the method of the present invention needs no stabilizing agent or molecular capping ligands in the solution to decrease the particle size and maintain dispersion colloidal stability.

The following may possibly be responsible for the colloidal stability of nanocrystals produced in pure water by the method of the present. Negative hydroxyl ions (OH⁻) are created at the same time that positive Ag ions (Ag⁺) and metallic Ag crystals are created by the electrochemical process. The OH⁻ anion behaves like a polarized dipole with negativity on the oxygen end and positivity on the hydrogen end. It therefore appears that the metallic Ag nanocrystals are colloidally stable at least partly due to the adsorption of hydroxyl OH⁻ anions (with the positive H end attracted to the Ag atom and the negative 0 end projecting out into the solution). The mutual electrostatic repulsion of the similar negative charges stabilizes the nanocrystals and keeps them from agglomerating. The negatively charged metallic nanocrystal particles can thus co-exist with the positively charged Ag ions. This is because thermal agitation of the ions is great enough to cause the ions to dissociate from the negatively charged surfaces of metal Ag particles.

Among other factors, the size of the metal nanocrystal colloids is likely determined by varying the current density (or voltage) that immediately affects the reduction potential of the cathode. The higher the over potential or over-voltage, which is defined as the deviation of the reduction potential from the equilibrium potential, the smaller becomes the maximum size of the metal nanocrystal nuclei in the electrolytic boundary layer. In accordance with the present invention, without the presence of surface active agents/stabilizers, the nanocrystal nuclei may be bounded with the HO⁻ anions to form a protective shell around them and thus prevent further growth due to ion addition or aggregation.

Suspensions with discrete nanometer Ag nanocrystals maintain a clear bright yellow color with larger particles turning the solution green or brown in color. The optical properties of nanocrystalline Ag are such that it absorbs strongly in the visible region due to surface plasmon resonance.

A typical process to produce Ag nanocrystals involves a reaction vessel containing a reaction liquid (typically distilled, deionized and 0.02 micron filtered) high-purity water heated to a target temperature (30-100° C.), Ag electrodes enclosed by dialysis membrane (DM) tubes, and a direct current (DC) power supply. The reaction vessel is brought up to the target temperature by immersion of the reaction vessel in a thermostatically controlled water or oil bath. Electrical current is applied to the Ag electrodes for a period of time sufficient to effect a deep golden yellow color. Such period of time is not particularly critical, and can vary from <5 to >60 minutes, usually about 20 to 30 minutes, depending on the amount of water used and spacing of the electrodes. Initially the current is 11 milliamps (ma) which reduces to 6 ma midway through the procedure as the power supply attempts to maintain constant voltage.

The present invention can be scaled up to much larger capacity vessels, perhaps hundreds of liters, and much higher voltages, perhaps several hundred volts. The parameters described herein are for test and prototype systems and methods, and the invention is not limited thereby.

Direct current is better than alternating current for the TEC synthesis of Ag nanocrystals. With alternating current, visible crystals and/or particles form quickly on electrodes and crystals aggregate into chains and fractal tree structure between two electrodes and micro-arcing could occur, resulting in a lot of large particles. Stirring of reaction solutions reduces formation of visible crystals significantly

Using direct current without dialysis membrane tube enclosed around electrodes, bright golden yellow solutions can be produced under appropriate conditions. However, large aggregates or crystal particles (originally formed on/near the electrode surfaces) tend to be mixed into the yellow solution containing nanocrystals. For dialysis membrane in “U” configuration (FIG. 1a), when not tied (cinched) to separate anode from cathode, nanocrystals are generally larger, ˜100 nm as compared when tied of ˜1 to 10 nm.

During experiments not using membranes, the larger, 100 nm crystals were formed along with the smaller ones. Filtering such nanocrystal samples with 200 nm filters usually resulted in samples with small, 1-10 nm crystals. Use of the membrane eliminates the need for filtering.

Reaction rate is temperature dependent and increases. At 3° C. almost no reaction was observed. Higher temperatures increase the nanocrystal formation rate. Moreover, reaction rate increases with decreasing electrode spacing and increasing voltage. Moreover, new, very smooth Ag wire electrode produces significantly higher reaction rates than the used and cleaned ones. Both produce small nanocrystals.

Concentration of the produced yellow nanocrystal sols has been attempted five ways and all but heating caused increases in nanocrystal size as evidenced by loss of golden yellow color of the solution and concurrent red shift in absorption spectrum:
1. Heating in hot bath reduced volume and retained original particle size.
2. Heating with sample directly on a hot plate reduced volume and also somewhat reduced original particle size.
3. Blowing nitrogen at room temperature to evaporate water produced significant increase in particle size.
4. Evaporation by vacuum pumping at room temperature produced significant increase in particle size.
5. Separation by putting nanocrystal sol into dialysis membrane produced significant increase in particle size.

When using DM, there seems to be an upper limit on nanoparticle concentration. Reuse of an entire assembly for 2 hours in pure filtered DI water resulted in extremely little production. Evidence for nanoparticles was only seen at most sensitive settings with absorption spectrometer. What happens is that the insides of the DM become darker over time and then Ag colored. It is possible that the membranes become barriers to the movement of Ag ions that would result in the formation of nanocrystals.

Presence of poly vinyl alcohol at 5 ppm in the reaction solutions produced concentrated suspension of small nanoparticles. Either side of the PVA concentration increased size. In a small reaction vessel with 5 ppm PVA at 98° C. and electrode spacing of 1 cm, no evidence of reaction at 1 volt for ½ hr. At 10 volts, evidence in 5 ppm PVA was seen in 8 minutes. Typical reactions in pure water seen with electrode spacing of about 2 cm is 45 min as compared with about min for 1 cm spacing. Volume was not constant; larger electrode spacing was used for larger volumes.

Some glass surfaces cause sample changes from the initially small nanocrystals to large ones (with change in color from golden to clear) within hours or days. The K-40 in normal glass may contribute to loss of small size. Literature on X-ray exposure to Ag nanocrystals forming larger ones supports this theory. Results were verified by DLS measurement and absorption spectra between 250-900 nm. Typical nanoparticles 20-50 nm give plasmon resonance about 420 nm. This shifts blue for smaller particles and red for larger. More narrow peak indicates small size distribution, wide peak indicates wider distribution. Loss of particles in 20-50 nm size range results in loss of 420 nm peak and enhancement of one about 600 nm.

Golden yellow solutions (containing nanocrystals of a few nanometer size) are stable for months if stored in small glass vials or low potassium scintillation glass vials.

Nanocrystalline Ag made in accordance with the present invention provides the following features:
stable chemically and colloidally, with long shelf-life (almost infinite) if stored properly in the right glass containers (low potassium also means the naturally radioactive isotope K-40 is also low).
clean surface, free from any organic capping molecules while maintaining colloidal stability in aqueous solution.
high purity in Ag (0.9999 purity is possible), absence of undesired side products such as organic contents or metal hydrides or boron impurities.
small nanosize (1-5 nm colloids), giving high specific surface area and thus the effectiveness for germicidal properties.
high particle concentration (90 vol/vol % can be achieved) by simple heated evaporation of background water.
straightforward isolation.
simple control of particle size by variation of synthesis conditions such as current density variation (by adjusting concentration of supporting electrolyte and/or stabilizing agent, varying voltage, temperature etc.)
nanocrystals of other metals such as Pt, Pd, Fe, Ni, Co, Ir, Rh, Cu, Au, etc. or nanocrystals of Ag alloyed with other metals can be synthesized, if the anode or counter electrode (cathode) are other metals (or alloys) than Ag. For example, Al, Ti, Sn, Ni, Cu, Pd, Ag, or Au can be readily dissolved metal anode.
less expensive relative to other methods such as metal evaporation and/or condensation and chemical reduction processes.

The scope of the present invention can be extended to various modifications, including but not limited to:
Various methods for heating such as infra-red direct heating or microwave heating that might impart modifications to the reaction kinetics of particles that are influenced by the local electromagnetic fields.
Various reaction vessels in term of size and arrangement.
Various geometry or materials of metal electrodes.
Various lasers might work to homogenize a nanocrystal sol sample.
Various metal nanocrystal synthesis, such as those listed herein.
Various membranes (with various MWCO DM or inorganic tube membrane).

The present invention is useful in various applications, including but not limited to the following:

Targeted therapy: when encapsulated with molecular self-assembly carrier systems like liposomes, the Ag-liposome package could be either injected into the blood stream or inhaled into the lung for targeted therapy. This kind of therapy may be critical to massive number of population infection of biological weapons and important for homeland security.

Ag bandages: Band-aid type dressings soaked with a few drops of Ag nanocrystals can conveniently be applied while wet. This will eradicate the bacteria in an open wound and prevent infection, possibly relieving pain in less than one hour.

Topical applications: In general, any medical condition of the skin subject to bacterial, viral or fungal infection are said to be treatable topically with silver. Specifically, following infections can be treated: ear, eye, sinus, yeast infection, sore throat and fungal infections of the skin (including athlete's foot ringworm, jock itch and toenail/fingernail fungus). Also included are conditions involving skin inflammation such as contact dermatitis. Nanocrystals of the present invention may be incorporated into creams, gels, solutions or other means for topical application.

Disinfection: Silver nanoparticles can be used alone or in the presence of chloride or iodide ions (that are said to enhance the biocidal action of silver) to disinfect substances from bacterial, viral and fungal contamination. In particular, pharmaceutical substances or their containers that are deteriorated by heat, radiation or other chemical disinfection methods may have their efficacy and/or shelf life enhanced by the use of silver nanoparticles directly in the storage containers at the time of filling. Similarly, stored or bottled water or other liquids can be maintained for long periods of time by incorporating silver nanoparticles in the container along with the liquid. Likewise, packaged foods may be disinfected by the use of silver nanoparticles alone or in the presence of chloride or iodide ions. This method can be used where heat or radiation disinfection are considered detrimental to the food quality.

Ingestion: some researchers have indicated that humans can ingest appropriate doses of Ag nanocrystal liquid sol to maintain dietary health. Ag nanocrystals are less affected than silver ions by the hydrochloric acid in the stomach and can be absorbed through the walls of the small intestine (or by sublingual absorption) and enter the bloodstream. Colloidally stable nanocrystal aqueous sol can also be injected into the blood stream and circulate. Bacteria and other pathogens in the bloodstream are said to be eradicated in contact with the Ag nanocrystals. The very high specific surface areas of nanocrystals are said to give the effective speed of pathogen eradication and solid crystal phase provide the required potency.

It should be noted that ionic Ag cannot survive inside the human body because it immediately combines with ubiquitous chloride ions to form Ag chloride (a form insoluble inside the body). Only the metallic Ag crystals survive to enter the blood stream where they are said to encounter and eradicate various pathogens.

Nebulization and Inhalation: Nanocrystal Ag aqueous sol can be applied to cure diseases such as sinus infection, Bronchitis, and upper respiratory infection including pneumonia.

Electronic Devices: Uniform Ag nanocrystals can be used as fundamental building blocks to self-assemble into 1D, 2D, and 3D nanostructures suitable for electronic device and sensors applications. The luminescent properties of nanocrystal Ag can be used for tracer/labeling applications.

The nanocrystals of the present invention represent a unique class of colloids, capable of exhibiting a variety of size-dependent optical and electronic properties that can be used in a variety of technologies, including coatings, environmental, chemical processing, medical, electronic, and sensing applications.

While there has been shown and described what are at present considered the preferred embodiments of the invention, it will be obvious to those skilled in the art that various changes and modifications can be prepared therein without departing from the scope of the inventions defined by the appended claims.

What is claimed is:

1. A composition of matter comprising a colloidally stable suspension of naked metal nanocrystals having an average particle size of no more than 100 nm.

2. A composition of matter in accordance with claim 1 wherein said naked metal nanocrystals have an average particle size of no more than 50 nm.

3. A composition of matter in accordance with claim 2 wherein said naked metal nanocrystals have an average particle size of no more than 20 nm.

4. A composition of matter in accordance with claim 3 wherein said naked metal nanocrystals have an average particle size of no more than 10 nm.

5. A composition of matter in accordance with claim 1 wherein said naked metal nanocrystals are essentially discrete.

6. A composition of matter in accordance with claim 1 wherein said metal comprises at least one metal selected from the group consisting of Ag, Au, Pd, Pt, Cu, Ru, Os, Ti, Fe, Cr, Co, Rh, Ni, Cd, Zn, Al, and any alloy comprising any of the foregoing.

7. A composition of matter in accordance with claim 6 wherein said nanocrystals consist essentially of Ag.

8. A method of making a colloidally stable suspension of naked metal nanocrystals comprising the steps of:
a. at least partly immersing a metallic sacrificial anode and a cathode into a body of essentially contaminant-free water, said metallic sacrificial anode comprising an essentially contaminant-free metal starting material for making nanocrystals; and
b. applying a voltage potential across said anode and said cathode to form a colloidally stable suspension of naked metal nanocrystals consisting essentially of metal from said metallic sacrificial anode.

9. A method in accordance with claim 8 wherein said metallic sacrificial anode comprises at least one metal selected from the group consisting of Ag, Au, Pd, Pt, Cu, Ru, Os, Ti, Fe, Cr, Co, Rh, Ni, Cd, Zn, Al, and any alloy comprising any of the foregoing.

10. A method in accordance with claim 9 wherein said metallic sacrificial anode consists essentially of Ag.

11. A method in accordance with claim 8 wherein said voltage potential is a DC voltage potential.

12. A method in accordance with claim 8 wherein said naked metal nanocrystals have an average particle size of no more than 100 nm.

13. A method in accordance with claim 12 wherein said naked metal nanocrystals have an average particle size of no more than 50 nm.

14. A method in accordance with claim 13 wherein said naked metal nanocrystals have an average particle size of no more than 20 nm.

15. A method in accordance with claim 14 wherein said naked metal nanocrystals have an average particle size of no more than 10 nm.

16. A method in accordance with claim 8 wherein said naked metal nanocrystals are essentially discrete.
